(19) United States
(12) Patent Application Publication
(10) Pub. No.: US 2023/0160080 A1
(43) Pub. Date: May 25, 2023

(54) METHODS OF FORMING AQUEOUS UREA UTILIZING CARBON DIOXIDE CAPTURED FROM EXHAUST GAS AT WELLSITE

(71) Applicant: Halliburton Energy Services, Inc., Houston, TX (US)

(72) Inventors: Philip D. NGUYEN, Houston, TX (US); Ronald Glen DUSTERHOFT, Houston, TX (US); Tatyana Vladimirov KHAMATNUROVA, Houston, TX (US)

(21) Appl. No.: 17/533,470

(22) Filed: Nov. 23, 2021

(51) Int. Cl. C25B 3/09; C25B 11/043; B01D 53/04; B01D 53/86; C25B 11/054; C25B 11/081; C25B 11/067

(52) U.S. Cl. CPC C25B 3/09; C25B 11/043; B01D 53/0423; B01D 53/8631; C25B 11/054; C25B 11/081; C25B 11/067; B01D 2256/22; B01D 2253/304; B01D 2253/1122; B01D 2253/1124; B01D 2258/02; B01D 2251/2067; B01D 2256/10

(57) ABSTRACT

A method includes collecting exhaust gas comprising carbon dioxide (CO₂) at a wellsite to provide a collected exhaust gas, separating CO₂ from the collected exhaust gas to provide a separated CO₂, and forming urea utilizing at least a portion of the separated CO₂. A system for carrying out the method is also provided.

### CROSS-REFERENCE TO RELATED APPLICATIONS

Not applicable.

### STATEMENT REGARDING FEDERALLY SPONSORED RESEARCH OR DEVELOPMENT

Not applicable.

### TECHNICAL FIELD

The present disclosure relates generally to systems and methods of sequestering carbon dioxide (CO₂). More specifically, this disclosure relates to collecting exhaust gas comprising CO₂ and forming urea utilizing at least a portion of the CO₂ in the collected exhaust gas. Still more specifically, this disclosure relates to collecting exhaust gas comprising CO₂, separating high purity CO₂ from the collected exhaust gas, and forming urea utilizing at least a portion of the high purity CO₂.

### BACKGROUND

Natural resources (e.g., oil or gas) residing in a subterranean formation can be recovered by driving resources from the formation into a wellbore using, for example, a pressure gradient that exists between the formation and the wellbore, the force of gravity, displacement of the resources from the formation using a pump or the force of another fluid injected into the well or an adjacent well. A number of wellbore servicing fluids can be utilized during the formation and production from such wellbores. For example, in embodiments, the production of fluid in the formation can be increased by hydraulically fracturing the formation. That is, a treatment fluid (e.g., a fracturing fluid) can be pumped down the wellbore to the formation at a rate and a pressure sufficient to form fractures that extend into the formation, providing additional pathways through which the oil or gas can flow to the well. Subsequently, oil or gas residing in the subterranean formation can be recovered or “produced” from the well by driving the fluid into the well. During production of the oil or gas, substantial quantities of produced water, which can contain high levels of total dissolved solids (TDS), and produced gas can also be produced from the well, and a variety of exhaust gases and flare gases conventionally sent to flare can be formed. For example, oil and gas wells produce oil, gas, and/or byproducts from subterranean formation hydrocarbon reservoirs. A variety of subterranean formation operations are utilized to obtain such hydrocarbons, such as drilling operations, completion operations, stimulation operations, production operations, enhanced recovery operations, and the like. Such subterranean formation operations typically use a large number of vehicles, heavy equipment, and other apparatus (collectively referred to as “machinery” herein) in order to achieve certain job requirements, such as treatment fluid pump rates. Such equipment may include, for example, pump trucks, sand trucks, cranes, conveyance equipment, mixing machinery, and the like. Many of these operations and machinery utilize combustion engines that produce exhaust gases (e.g., including carbon dioxide/greenhouse gas emissions) that are emitted into the atmosphere.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic flow diagram of a method, according to embodiments of this disclosure;

FIG. 2 is a schematic of a system, according to one or more embodiments of the present disclosure;

FIG. 3 is a schematic of a system, according to one or more embodiments of this disclosure;

FIG. 4 is a schematic of a urea production apparatus, according to one or more embodiments of the present disclosure;

FIG. 5 is a schematic of an electrocatalyst suitable for use in the urea production apparatus of FIG. 4, according to one or more embodiments of the present disclosure; and

FIG. 6 is a schematic of a plurality of machinery that may be located and operated a wellsite for performing a subterranean formation operation and may produce exhaust gas comprising CO₂, according to one or more embodiments of the present disclosure.

### DETAILED DESCRIPTION

It should be understood at the outset that although an illustrative implementation of one or more embodiments are provided below, the disclosed systems and/or methods can be implemented using any number of techniques, whether currently known or in existence. The disclosure should in no way be limited to the illustrative implementations, drawings, and techniques below, including the exemplary designs and implementations illustrated and described herein, but can be modified within the scope of the appended claims along with their full scope of equivalents.

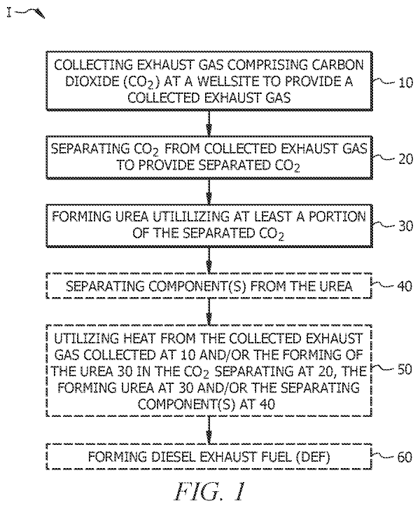

A method of this disclosure will now be described with reference to FIG. 1, which is a schematic flow diagram of a method I according to one or more embodiments of this disclosure. As seen in FIG. 1, method I includes collecting exhaust gas comprising carbon dioxide (CO₂) at a wellsite to provide a collected exhaust gas at 10, separating CO₂ from the collected exhaust gas to provide a separated CO₂ at 20, and forming urea utilizing at least a portion of the separated CO₂ at 30. A method I of this disclosure can further comprise: at 40, separating one or more components from the urea; at 50, utilizing heat from the collected exhaust gas collected at 10 and/or the forming of the urea at 30 in the separating CO₂ at 20, the forming of urea at 30 and/or the separating components at 40; and/or, at 60, forming diesel exhaust fluid (DEF). Although depicted in a certain order in FIG. 1, in embodiments, one or more of steps 10 to 60 can be absent, and/or the one or more of steps 10 to 60 can be performed more than once and/or in a different order than described herein or depicted in the embodiment of FIG. 1.

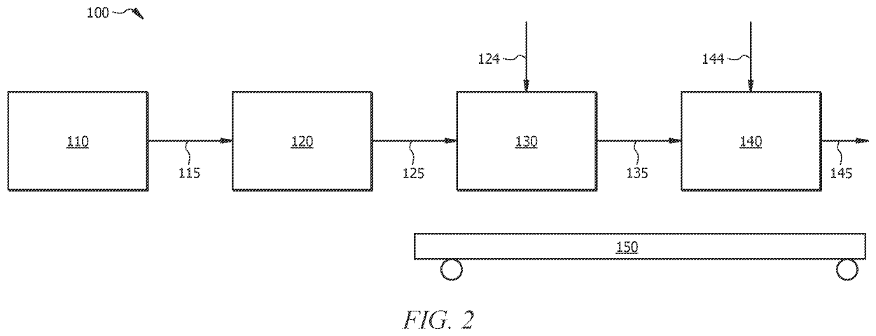

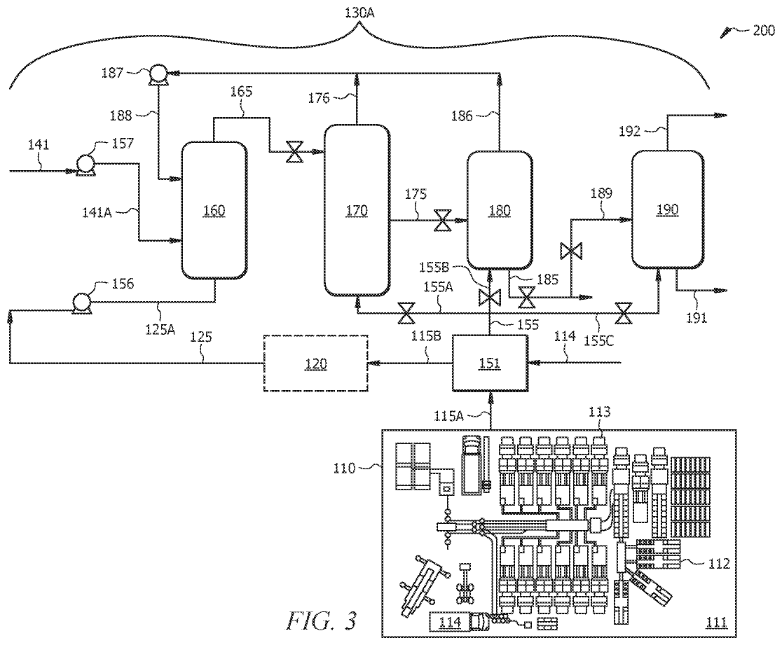

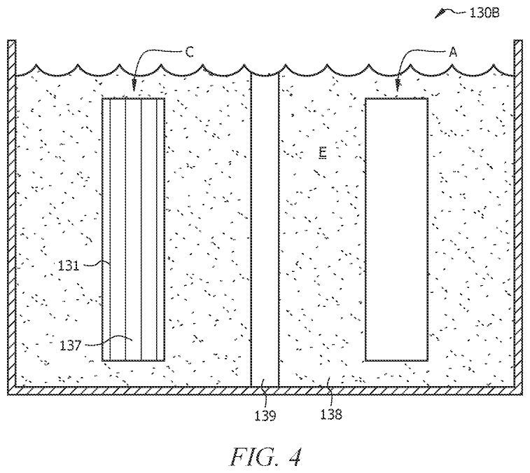

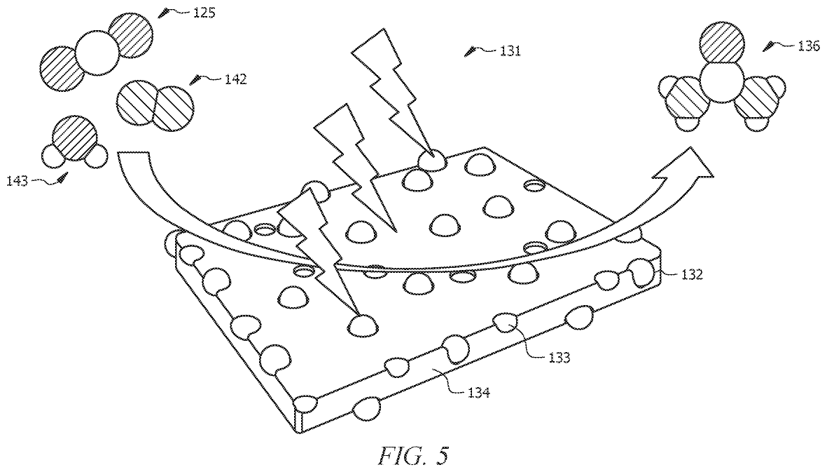

The method of this disclosure will now be detailed and a system for carrying out the method according to embodiments of this disclosure described with reference to FIG. 2, which is a schematic of a system 100 according to one or more embodiments of this disclosure, FIG. 3, which is a schematic of a system, 200 according to one or more embodiments of this disclosure, FIG. 4, which is a schematic of a urea production apparatus 130B disparate from urea production apparatus 130A of system 200 of FIG. 3, and FIG. 5, which is a schematic of an electrocatalyst 131 suitable for use in the urea production apparatus 130B of FIG. 4, according to one or more embodiments of the present disclosure.

With reference now to FIG. 2, system 100 comprises: an exhaust gas collection system 110 configured for collecting exhaust gas 115 comprising carbon dioxide (CO₂) at a wellsite 111 (FIG. 3) to provide a collected exhaust gas (e.g., step 10 of FIG. 1); a CO₂ separation apparatus 120 configured for separating CO₂ from the collected exhaust gas 115 to provide a separated CO₂ 125 (e.g., step 20 of FIG. 1); and a urea production and/or purification apparatus 130 (referred to hereinafter simply as “urea production apparatus 130”) configured for forming urea (and optionally separating one or more components therefrom) to provide a urea product 135 comprising urea utilizing at least a portion of the separated CO₂ 125 (e.g., step 30 of FIG. 1). As depicted in FIG. 2, system 100 can further comprise a diesel exhaust fluid (DEF) production apparatus 140 configured to form DEF (e.g., by diluting the urea product 135 with dilution water 144 to form DEF 145).

As noted above with reference to FIG. 1, method I includes collecting exhaust gas comprising carbon dioxide (CO₂) at a wellsite to provide a collected exhaust gas 115 at 10. Collecting exhaust gas at 10 can be effected via exhaust gas collection apparatus 110. Exhaust gas collection apparatus 110 can include and/or obtain the collected exhaust gas 115 from field operating equipment 112 (FIG. 3) at a wellsite 111. The field operating equipment 112 can comprise one or more vehicles (e.g., diesel trucks, cars, etc.), pumps (e.g., hydraulic pumps, fracturing pumps, etc.), or other equipment at a wellsite 111 that produces an exhaust gas comprising CO₂ from which collected exhaust gas 115 is obtained. Exhaust gas collection apparatus 110 can further comprise piping configured to combine the exhaust gas from a plurality of the field operating equipment 112 and introduce it to CO₂ separation apparatus 120, storage apparatus to store the collected exhaust gas 115 prior to introduction into CO₂ separation apparatus 120, or a combination thereof. Collecting the collected exhaust gas 115 comprising CO₂ at 10 can be performed by piping exhaust gas from one or more pieces of field operating equipment or machinery 112 at a wellsite 111 to provide the collected exhaust gas 115.

The exhaust gas comprising CO₂ collected at step 10 can include greater than or equal to about 0.04, 0.1, 0.5, 1, 2, 3, 4, 5, 6, 7, 8, 9, 10, 20, 30, 40, 50, 60, 70, 80, 90, 95, or 100 volume percent (vol. %) CO₂. By way of examples, the collected exhaust gas comprising CO₂ 115 can include a waste gas, or one or more components thereof, produced at the wellsite 111 or another jobsite, such as, without limitation, one or more wellsites or industrial plants. The one or more industrial plants can include, without limitation, a cement plant, a chemical processing plant, a mechanical processing plant, a refinery, a steel plant, a power plant (e.g., a gas power plant, a coal power plant, etc.), or a combination and/or a plurality thereof. In embodiments, the exhaust gas comprising CO₂ 115 comprises a waste gas that is a product of fuel combustion, for example, the product of an internal combustion engine, or a gas fired turbine engine, such as, for example, from a microgrid having electric pumps. In embodiments, the internal combustion engine includes an engine fueled by diesel, natural gas (e.g., methane), gasoline, or a combination thereof (e.g., a diesel engine, or a hybrid engine that is fueled by diesel and natural gas). The collected exhaust gas comprising CO₂ 115 can be produced at the wellsite 111 and/or another jobsite. A plurality of machinery 112 can be located and operated at a wellsite 111 for performing a subterranean formation operation, according to one or more embodiments of the present disclosure, and the collected exhaust gas comprising CO₂ 115 can, in embodiments, be obtained therefrom. For example, the exhaust gas comprising CO₂ from which the collected exhaust gas 115 can be produced at the wellsite 111 from machinery 112 used to perform a wellbore servicing operation. The machinery may include one or more internal combustion or other suitable engines that consume fuel to perform work at the wellsite 111 and produce exhaust gas comprising CO₂ from which collected exhaust gas 115 is collected.

The wellbore 101 at wellsite 111 may be a hydrocarbon-producing wellbore (e.g., oil, natural gas, and the like) or another type of wellbore for producing other resources (e.g., mineral exploration, mining, and the like). Machinery 112 typically associated with a subterranean formation operation related to a hydrocarbon producing wellbore, and from which the exhaust gas comprising CO₂ can be produced, can be utilized to perform such operations as, for example, a cementing operation, a fracturing operation, or other suitable operation where equipment is used to drill, complete, produce, enhance production, and/or work over the wellbore. Other surface operations may include, for example, operating or construction of a facility.

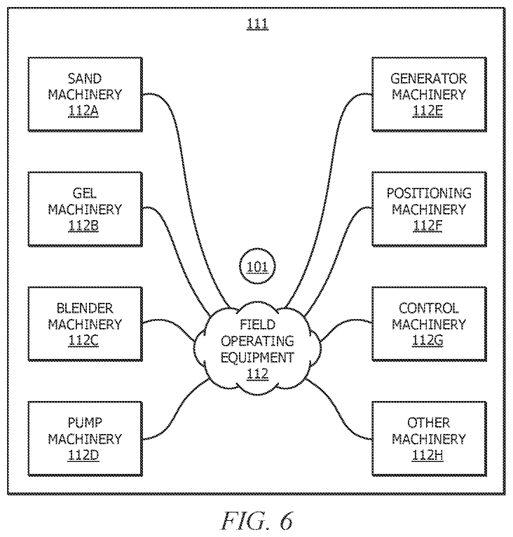

As depicted in FIG. 6, which is a schematic of a plurality of machinery 112 that may be located and operated a wellsite 111 for performing a subterranean formation operation and may produce exhaust gas comprising CO₂ from which collected exhaust gas 115 is collected, according to one or more embodiments of the present disclosure, the machinery 112 from which the exhaust gas comprising CO₂ can be produced, in embodiments, can include sand machinery 112A, gel machinery 112B, blender machinery 112C, pump machinery 112D, generator machinery 112E, positioning machinery 112F, control machinery 112G, and other machinery 112H. The machinery 112 may be, for example, truck, skid or rig-mounted, or otherwise present at the wellsite 111, without departing from the scope of the present disclosure. The sand machinery 112A may include transport trucks or other vehicles for hauling to and storing at the wellsite 111 sand for use in an operation. The gel machinery 112B may include transport trucks or other vehicles for hauling to and storing at the wellsite 111 materials used to make a gelled treatment fluid for use in an operation. The blender machinery 112C may include blenders, or mixers, for blending materials at the wellsite 111 for an operation. The pump machinery 112D may include pump trucks or other vehicles or conveyance equipment for pumping materials down the wellbore 101 for an operation. The generator machinery 112E may include generator trucks or other vehicles or equipment for generating electric power at the wellsite 111 for an operation. The electric power may be used by sensors, control machinery, and other machinery. The positioning equipment 112F may include earth movers, cranes, rigs or other equipment to move, locate or position equipment or materials at the wellsite 111 or in the wellbore 101.

The control machinery 112G may include an instrument truck coupled to some, all, or substantially all of the other equipment at the wellsite 111 and/or to remote systems or equipment. The control machinery 112G may be connected by wireline or wirelessly to other equipment to receive data for or during an operation. The data may be received in real-time or otherwise. In another embodiment, data from or for equipment may be keyed into the control machinery.

The control machinery 112G may include a computer system for planning, monitoring, performing or analyzing the job. Such a computer system may be part of a distributed computing system with data sensed, collected, stored, processed and used from, at or by different equipment or locations. The other machinery 112H may include equipment also used at the wellsite 111 to perform an operation.

In other examples, the other machinery 112H may include personal or other vehicles used to transport workers to the wellsite 111 but not directly used at the wellsite 111 for performing an operation.

Many if not most of these various machinery 112 at the wellsite 11 accordingly utilize a diesel or other fuel types to perform their functionality. Such fuel is expended and exhausted as exhaust gas, such as exhaust gas including CO₂. The embodiments described herein provide a system and method for collecting, converting to urea, and, thus, sequestering CO₂ from such machinery 112 located and operated at a wellsite 111, thus reducing atmospheric CO₂ emissions, while reducing material and time costs. It is to be appreciated that other configurations of the wellsite 111, including other machinery 112 at the wellsite 111 or another jobsite, may be employed, without departing from the scope of the present disclosure. Although a number of various machinery 112 at a jobsite (e.g., a wellsite 111) have been mentioned, many other machinery may utilize diesel or other fuel that creates exhaust gas including CO₂ that may conventionally be exhausted into the atmosphere, but herein utilized to form urea as described herein.

In some embodiments, the present disclosure provides capturing exhaust gas comprising CO₂ 115 from such machinery located and operated at a wellsite 111 and utilizing such exhaust gas to form urea as detailed herein.

Although described hereinabove with reference to a wellsite 111, the source of the collected exhaust gas comprising CO₂ 115 that is collected at step 10 of the method I can be any convenient CO₂ source. The CO₂ source can be a gaseous CO₂ source. This gaseous CO₂ may vary widely, ranging from air, industrial waste streams, etc. As noted above, the gaseous CO₂ can, in certain instances, comprise an exhaust waste product from an industrial plant. The nature of the industrial plant may vary in these embodiments, where industrial plants of interest include power plants, chemical processing plants, and other industrial plants that produce exhaust gas comprising CO₂ as a byproduct. By waste stream is meant a stream of gas (or analogous stream) that is produced as a byproduct of an active process of the industrial plant, e.g., an exhaust gas. The gaseous stream may be substantially pure CO₂ or a multi-component gaseous stream that includes CO₂ and one or more additional gases. Multi-component gaseous streams (containing CO₂) that may be employed as a CO₂ source in embodiments of the subject methods include both reducing, e.g., syngas, shifted syngas, natural gas, and hydrogen and the like, and oxidizing condition streams, e.g., flue gases from combustion. Particular multi-component gaseous streams of interest that may be treated according to the subject invention include: oxygen containing combustion power plant flue gas, turbo charged boiler product gas, coal gasification product gas, shifted coal gasification product gas, anaerobic digester product gas, wellhead natural gas stream, reformed natural gas or methane hydrates, and the like.

As noted above, in embodiments, the collected exhaust gas comprising CO₂ 115 comprises greater than or equal to about 5, 6, 7, 8, 9, 10, 15, 20, 25, 30, 40, 50, 60, 70, 80, 90, 95, 96, 97, 98, 99, or 100 volume percent (vol %) CO₂. In embodiments, the exhaust gas comprising CO₂ 115 comprises primarily CO₂ (e.g., greater than or equal to about 50, 60, 70, 80, 90, 95, 96, 97, 98, 99, or 100 volume percent (vol %) CO₂). For example, when the exhaust gas comprising CO₂ 115 is obtained from a waste gas produced at a different jobsite (e.g., at an another jobsite) than the wellsite 111, CO₂ can be separated from the waste gas in order to reduce a volume of gas to be transported to the wellsite 111. For example, when the exhaust gas includes a flue gas from a power plant, which typically contains from about 7 to about 10 vol. % CO₂, the method I can further include transporting the exhaust gas comprising CO₂ (or a waste gas from which the exhaust gas comprising CO₂ 115 is obtained) from the another jobsite at which the waste gas is obtained to the wellsite 111. In embodiments, the method I can further include separating the exhaust gas from the waste gas comprising CO₂, to reduce a volume of gas, e.g., for transport. Although the separating of the exhaust gas comprising CO₂ from the waste gas can be performed at the wellsite 111 (e.g., after transport of the waste gas from the another jobsite at which the waste gas is obtained and/or produced to the wellsite 111), to facilitate transportation, the separating of the collected exhaust gas comprising CO₂ 115 from the waste gas can be performed at the another jobsite at which the waste gas is produced and/or obtained and, subsequently, the collected exhaust gas comprising CO₂ 115 can be transported to the wellsite 111.

As noted above, method I comprises, at 20, separating CO₂ from the collected exhaust gas to provide a separated CO₂ 125. Separating the CO₂ from the collected exhaust gas 115 at 20 can comprise separating substantially pure CO₂ from the collected exhaust gas 115. That is, in embodiments, the separated CO₂ 125 is substantially pure CO₂. The substantially pure CO₂ (and the substantially pure separated CO₂ 125) can include greater than or equal to about 90, 95, 96, 97, 98, 99, 99.5, 99.8, 99.9, or 100 vol % CO₂. Separating CO₂ from the collected exhaust gas 115 to provide a separated CO₂ 125 at 20 can be effected by CO₂ separation apparatus 120. CO₂ separation apparatus 120 can comprise any apparatus operable to provide high purity (e.g., greater than or equal to 50, 60, 70, 80, 90, 95, 96, 97, 98, 98.5, 99, 99.5, 99.9, 99.99, or substantially 100 volume percent (vol %) CO₂ from the collected exhaust gas 115. In embodiments, CO₂ separation apparatus 120 can operate by separating via amine absorption, calcium oxide (CaO) absorption, filtration, packed bed, another technique, or a combination thereof. In embodiments, CO₂ separation apparatus 120 comprises batch reactor, continuous reactor, packed-bed column, fluidized bed column, or a combination thereof.

As noted above, method 1 comprises, at 30, forming urea utilizing at least a portion of the separated CO₂ 125. With reference now to FIG. 3, forming urea at step 30 can comprise reacting the separated CO₂ 125 with ammonia (NH₃) 141 to form ammonium carbamate (e.g., in ammonium carbamate solution 165) and decomposing the ammonium carbamate in ammonium carbamate solution 165 to form the urea (e.g., in aqueous urea solution 175); or, with reference to FIG. 4, forming urea at step 30 can comprise reacting the separated CO₂ 125 directly with nitrogen (N₂) gas 142 to form the urea 136.

Forming urea at 30 can be effected via urea production apparatus 130. Urea production apparatus 130 can comprise any apparatus operable to produce a urea product 135 comprising urea from at least a portion of the separated CO₂ 125. For example, and with reference to FIG. 3, in embodiments, urea production apparatus 130 comprises an NH₃—CO₂ reactor 160 configured for reacting the separated CO₂ 125 with ammonia (NH₃) 141 to form an ammonium carbamate solution 165 comprising ammonium carbamate and an ammonium carbamate decomposition reactor 170 configured for decomposing the ammonium carbamate to form the urea product 135 comprising urea. By way of further example, and with reference to FIG. 4, in embodiments, urea production apparatus 130 comprises an electrocatalytic reactor 130 configured for forming the urea product 135 by reacting the separated CO₂ 125 directly with nitrogen gas 142. Utilization of urea production apparatus 130 other than that depicted in FIG. 3, FIG. 4, and FIG. 5 to form the urea at step 30 is intended to be within the scope of this disclosure.

In embodiments, described now with reference to system 200 of FIG. 3, the substantially pure CO₂ 125 separated from the collected exhaust gas 115 is compressed to react with liquid ammonia 141 in NH₃—CO₂ reactor 160, wherein an exothermic reaction occurs between CO₂ and NH₃ to form ammonium carbamate (NH₂COONH₄) as shown in Equation (1):

2NH₃+CO₂→NH₂COONH₄ Eq. (1)

The heat generated from the exothermic reaction of Equation (1) (e.g., about 380° F. and 150 atm) can be optionally captured by a heat exchanger (e.g., heat exchanger 150) to produce steam 155 and provide a heat source for heating the ammonium decomposition reactor 170 which can function as a distillation column to decompose the ammonium carbamate in ammonium carbamate solution 165 into an aqueous urea solution 175 via Equation (2) as the temperature and pressure in the ammonium carbamate decomposition reactor 170 are decreased (e.g., to about 275° F. and 35 atm).

NH₂COONH₄→NH₂CONH₂+H₂O Eq. (2)

The distillation process in ammonium carbamate decomposition reactor 170 can also purify the produced aqueous urea solution 175 by removing a stream 176 comprising water, unreacted NH₃, CO₂, and ammonium carbamate (e.g., at separating component(s) step 40 of method I of FIG. 1). Stream 176 can be recycled to NH₃—CO₂ reactor 160.

The aqueous urea solution 175 can be transferred to a unreacted component removal reactor 180 (also referred to herein as an “unwanted component removal apparatus 180”, as reactor 180 removes non-urea components of aqueous solution 170, not just unreacted reactants NH₃ and CO₂), which can act as a flash chamber/condenser in embodiments, allowing the unreacted component removal apparatus 180 to be depressurized to, for example, ambient pressure while heating to further decompose any unreacted ammonium carbamate to NH₃ and CO₂, as shown in Equation (3).

NH₂COONH₄→2NH₃+CO₂ Eq. (3).

Like stream 176 from ammonium carbamate decomposition reactor 170, a stream 186 from unreacted component removal reactor 180 comprising water and unreacted NH₃ and CO₂ can also be recycled to NH₃—CO₂ reactor 160, thus increasing the concentration of urea in the concentrated aqueous urea 185 relative to a concentration of urea in the aqueous urea solution 175. In embodiments, the concentrated aqueous urea solution 185 comprises greater than or equal to about 80, 85, or 90, or from about 80 to 100 weight percent (wt %) urea. In embodiments, the concentrated urea solution 185 can be transformed into semi-solid, dry solids, or granulated urea 191 by additional drying of concentrated aqueous urea solution 185 in a drying apparatus 190. In embodiments, drying apparatus 190 can comprise a vacuum evaporator operated under vacuum with temperatures of greater than or equal to about 250° F.

In embodiments, the heat of the collected exhaust gas 115 collected from the equipment 110 operating at wellsite 111 or heat provided by the reaction of Eq. (1) in NH₃—CO₂ reactor 160 can be utilized to attain operating temperatures in the ammonium carbamate decomposition reactor 170, unreacted component removal reactor 180, and/or drying apparatus 190, as described further hereinbelow.

In embodiments such as depicted in FIG. 3, the urea production apparatus 130 (FIG. 2) comprises urea production apparatus 130A comprising NH₃—CO₂ reactor 160 and ammonium carbamate decomposition reactor 170 configured for decomposing the ammonium carbamate to provide urea.

With reference now to FIG. 1 and FIG. 3, in embodiments, forming urea at step 30 comprises reacting the at least the portion of the separated CO₂ 125 with ammonia 141 to form ammonium carbamate and decomposing the ammonium carbamate to form the urea. In such embodiments, forming urea at step 30 can further comprise: compressing the at least a portion of the separated CO₂ 125 to provide a compressed CO₂ 125A; reacting the compressed CO₂ 125A with the ammonia 141 to form an ammonium carbamate solution 165 comprising the ammonium carbamate; and decomposing the ammonium carbamate to provide an aqueous urea solution 175 comprising the urea.

Thus, as depicted in FIG. 3, system 200 can further comprise a compressor 156 configured for compressing the at least a portion of the separated CO₂ 125 to provide a compressed CO₂, 125A. In such embodiments, the NH₃—CO₂ reactor 160 can be configured for reacting the compressed CO₂ 125A with the liquid ammonia 141 via an exothermic reaction to form the ammonium carbamate. In embodiments, compressor 156 is operable to compress the at least the portion of the separated CO₂ 125 to a pressure of greater than or equal to about 130, 140, or 150 atmospheres (13.2, 14.2, or 15.2 MPa). Method I can comprise, at forming urea step 30, compressing the at least the portion of the separated CO₂ 125 (e.g., in compressor 156) to provide compressed CO₂ 125A having a pressure of greater than or equal to about 130, 140, or 150 atmospheres (13.2, 14.2, or 15.2 MPa). Reacting the compressed CO₂ 125A and ammonia 141 to form the ammonium carbamate solution 165 can comprise introducing the compressed CO₂ 125A and liquid ammonia 141 into NH₃—CO₂ reactor 160, whereby the compressed CO₂ 125A and the liquid NH₃ 141 react via the exothermic reaction of Eq. (1) to produce the ammonium carbamate. Ammonium carbamate solution 165 comprising the ammonium carbamate can be removed from NH₃—CO₂ reactor 160 and introduced into ammonium carbamate decomposition reactor 170.

NH₃—CO₂ reactor 160 is configured for reacting the separated CO₂ 125 with liquid ammonia comprising the NH₃ 141 to form the ammonium carbamate solution 165 comprising ammonium carbamate. NH₃—CO₂ reactor 160 can have an operating temperature in a range of from about 350° F. to about 390° F. (from about 176.7° C. to about 198.9° C.), from about 360° F. to about 390° F. (from about 182.2° C. to about 198.9° C.), from about 370° F. to about 390° F. (from about 176.7° C. to about 198.9° C.), or greater than or equal to about 350° F., 360° F., 370° F., 380° F., or 390° F. (greater than or equal to about 176.7° C., 182.2° C., 187.8° C., 193.3° C., or 198.8° C.), and/or a pressure in a range of from about 130 atmospheres (atm) to about 160 atm (from about 13.2 MPa to about 16.2 MPa), from about 140 atm to about 160 atm (from about 14.2 MPa to about 16.2 MPa), from about 150 atm to about 160 atm (from about 15.2 MPa to about 16.2 MPa), or greater than or equal to about 130, 140, 150, 160, or 170 atm (greater than or equal to about 13.2, 14.2, 15.2, 16.2, or 17.2 MPa).

As noted above and detailed further hereinbelow, method I can further comprise using heat produced by the exothermic reaction in the NH₃—CO₂ reactor 160 to produce steam 155 (e.g., via heat exchanger 150 or another heat exchanger). The steam can be utilized as a heat source for decomposing the ammonium carbamate utilized in forming urea at step 30, or elsewhere throughout system 200.

Decomposing the ammonium carbamate during the forming of urea at 30 utilizing system 200 of FIG. 3 can comprise introducing the ammonium carbamate solution 165 comprising ammonium carbamate into ammonium carbamate decomposition reactor 170. As noted above, in ammonium carbamate decomposition reactor 170, the ammonium carbamate in ammonium carbamate solution 165 decomposes to provide the urea of the aqueous urea solution 175 via a reduction in the temperature and the pressure of the ammonium carbamate (e.g., in ammonium carbamate decomposition reactor 170). Decomposing the ammonium carbamate during forming of the urea at step 30 can further comprise reducing the temperature and pressure of the ammonium carbamate in the ammonium carbonate decomposition reactor 170 to a temperature in a range of from about 250° F. to about 280° F. (from about 121.1° C. to about 137.8° C.), from about 260° F. to about 280° F. (from about 126.7° C. to about 137.8° C.), from about 270° F. to about 280° F. (from about 132.2° C. to about 137.8° C.), or less than or equal to about 250° F., 260° F., 270° F., 280° F., or 290° F. (less than or equal to about 121.1° C., 126.7° C., 132.2° C., 137.8° C., or 143.3° C.), and/or a pressure in a range of from about 20 atmospheres (atm) to about 50 atm (from about 2.1 MPa to about 5.1 MPa), from about 25 atm to about 45 atm (from about 2.5 MPa to about 4.6 MPa), from about 30 atm to about 50 atm (from about 3.0 MPa to about 5.1 MPa), or less than or equal to about 50, 45, 40, 35, or 30 atm (less than or equal to about 5.1, 4.6, 4.1, 3.5, or 3.0 MPa).

Accordingly, urea production apparatus 130A can further comprise ammonium carbamate decomposition reactor 170. Ammonium carbamate solution 165 comprising ammonium carbamate, water, unreacted NH₃ and unreacted CO₂ can be introduced into ammonium carbamate decomposition reactor 170. The ammonium carbamate decomposition reactor 170 is configured for decomposing the ammonium carbamate to form an aqueous urea solution 175 comprising the urea. An ammonium carbamate decomposition reactor outlet stream 176 comprising water and unreacted CO₂ and NH₃ can be removed from ammonium carbamate reactor decomposition reactor 170. Ammonium carbamate decomposition reactor 170 can be operable to decompose the ammonium carbamate to provide the aqueous urea solution 175 via a reduction in the temperature and pressure of the ammonium carbamate in the ammonium carbamate decomposition reactor 170. In embodiments, the ammonium carbamate decomposition reactor 170 comprises and/or is operable as a distillation column.

In embodiments, the ammonium carbamate decomposition reactor 170 effects the decomposing of the ammonium carbamate in ammonium carbamate solution 175 via a reduction of the temperature and pressure of the ammonium carbamate therein to a temperature in a range of from about 250° F. to about 280° F. (from about 121.1° C. to about 137.8° C.), from about 260° F. to about 280° F. (from about 126.7° C. to about 137.8° C.), from about 270° F. to about 280° F. (from about 132.2° C. to about 137.8° C.), or less than or equal to about 250° F., 260° F., 270° F., 280° F., or 290° F. (less than or equal to about 121.1° C., 126.7° C., 132.2° C., 137.8° C., or 143.3° C.), and/or a pressure in a range of from about 20 atmospheres (atm) to about 50 atm (from about 2.1 MPa to about 5.1 MPa), from about 25 atm to about 45 atm (from about 2.5 MPa to about 4.6 MPa), from about 30 atm to about 50 atm (from about 3.0 MPa to about 5.1 MPa), or less than or equal to about 50, 45, 40, 35, or 30 atm (less than or equal to about 5.1, 4.6, 4.1, 3.5, or 3.0 MPa). The ammonium carbamate decomposition reactor 170 can further comprise an outlet for a stream 176 comprising water, unreacted NH₃ and CO₂, and (e.g., undecomposed) ammonium carbamate. The ammonium carbamate decomposition reactor 170 can be fluidly connected with the NH₃—CO₂ reactor 160, whereby the stream 176 comprising water, unreacted NH₃ and CO₂, and undecomposed ammonium carbamate can be recycled to the NH₃—CO₂ reactor 160, to increase a conversion to urea by the system 200.

In embodiments such as depicted in FIG. 4 and FIG. 5, the urea production apparatus 130 (FIG. 2) comprises an electrocatalytic reactor comprising a flow reactor cell 130B. A system of this disclosure can thus be as depicted in FIG. 3, with urea production apparatus 130B of FIG. 4 utilized in place of urea production apparatus 130A of FIG. 3. The electrocatalytic reactor of urea production apparatus 130B is configured for forming urea 136 by reacting the separated CO₂ 125 directly with nitrogen gas 142 in water 143 to form the urea 136. Electrocatalytic reactor 130B can be configured for forming the urea 136 by converting nitrogen gas 142 and the at least the portion of the separated CO₂ 125 directly into urea 136 in water 143 via an electrocatalytic reaction. In embodiments, electrocatalytic reactor 130B is configured to effect the electrocatalytic reaction at about ambient temperature and pressure (e.g., a temperature of about 77° F. (21° C.) and atmospheric pressure (101 kPa)).

As depicted in FIG. 5, electrocatalytic reactor 130B can comprise an electrocatalyst 131 comprising palladium-copper (Pd—Cu) nanoparticles 132 on titanium dioxide (TiO₂) nanosheets 134, having oxygen vacancies 133. Electrocatalytic reactor 130B can comprise a flow reactor cell comprising a cathode C of carbon paper 137 loaded with the catalyst 131 and a nickel based anode A, separated by a membrane 139 in a chamber 138 filled with an aqueous (e.g., potassium bicarbonate) electrolyte E. In such embodiments, urea 136 is formed by pumping the N₂ gas 142 and the at least the portion of the separated CO₂ 125 gas through the flow reactor cell 130B so both the nitrogen gas 142 and the CO₂ gas 125 are adsorbed on the catalyst 131 and react to produce the urea 136.

Thus, in embodiments, forming urea at 30 can be effected directly at ambient conditions. For example, with reference to electrocatalytic urea production apparatus 130B of FIG. 4 and electrocatalyst 131 of FIG. 5, forming urea at step 30 can comprise coupling N₂ gas 142 and separated CO₂ 125 in H₂O 143 to directly synthesize urea 136 under ambient conditions. In such embodiments, electrocatalytic reaction can be applied to convert nitrogen gas 142 and substantially pure separated CO₂ 125 directly into urea 136 aqueous solution (e.g., the presence of water 143) via electrocatalytic reaction. The electrocatalytic reaction can occur at ambient temperature and pressure (e.g., a temperature of about 77° F. (21° C.) and a pressure of about atmospheric pressure (101 kPa)).

The electrocatalytic reaction can employ an electrocatalyst 131 (FIG. 5) comprising or consisting of palladium-copper nanoparticles 132 on titanium dioxide nanosheets 134, or another electrocatalyst. The electrocatalytic reaction can be effected in a flow reactor cell 130B containing a cathode C made of carbon paper 137 or another cathode material loaded with the electrocatalyst 131 (or other electrocatalyst) and a nickel-based anode A or another anode, separated by a membrane 139 in a chamber 138 filled with an aqueous potassium bicarbonate electrolyte E or another electrolyte. The electrodes, separated by a membrane 139, are positioned in a chamber 138 filled with an electrolyte E (e.g., an aqueous potassium bicarbonate electrolyte). The N₂ 142 and CO₂ 125 gases are pumped through the flow reactor cell 130B so that both gases are adsorbed on the electrocatalyst 131 and react to produce urea 136. Forming the urea at step 30 can include pumping the N₂ gas 142 and the at least the portion of the separated CO₂ gas 125 through the flow reactor cell 130B so both the nitrogen gas 142 and the CO₂ gas 125 are adsorbed on the catalyst 131 and react to produce the urea 136.

Without being limited by theory, nitrogen 142 can promote the reduction of CO₂ 125 on the catalyst 131 surface to produce carbon monoxide (CO). The CO can then react with N₂ 142 to generate intermediate species. Further interactions between CO and these intermediate species hydrogenate N₂ 142 and create C-N bonds, thereby forming urea 136. The titanium dioxide support 134 can play a key role in the urea 136 synthesis by stabilizing the intermediates. Increasing the electrolyte E concentration and flow rate can be utilized to improves the efficiency of the electrocatalytic reaction system 130B.

As noted above, method I can further comprise, at 40, separating one or more components from the urea. For example, water, unreacted NH₃, unreacted CO₂, ammonium carbamate (e.g., undecomposed ammonium carbamate), or a combination thereof can be removed from aqueous urea solution 175 at 40.

With reference back to system 200 of FIG. 3, in embodiments, separating components from the urea at 40 can further comprise removing water, and unreacted NH₃ and CO₂ stream 186 from the aqueous urea solution 175 to provide a concentrated aqueous urea 185. In embodiments, urea production apparatus 130 can include unwanted component removal apparatus configured to remove one or more unwanted components (e.g., water, unreacted reactants (e.g., NH₃, CO₂, N₂) from the urea produced in the urea production apparatus 130. For example, urea production apparatus 130A of system 200 can further comprise an unreacted component removal apparatus 180 configured for removing a stream 186 comprising water and unreacted NH₃ and CO₂ from the aqueous urea solution 175 to provide a concentrated aqueous urea 185.

As noted above, removing the stream 186 comprising additional water and unreacted NH₃ and CO₂ can be effected via unreacted component removal reactor 180 (e.g., a flash chamber). Aqueous urea solution 175 provided in the ammonium carbamate decomposition reactor 170 can thus be introduced into unreacted component removal reactor 180. Unreacted component removal reactor 180 is operable to provide concentrated aqueous urea 185. Concentrated aqueous urea 185 has a higher concentration of urea than aqueous urea solution 175 introduced thereto. In embodiments, the unreacted component removal apparatus 180 comprises a flash chamber. Stream 186 comprising water, unreacted NH₃ and/or unreacted CO₂ can be removed from unreacted component removal apparatus 180. Unreacted component removal apparatus 186 can be fluidly connected with NH₃—CO₂ reactor 160, such that stream 186 comprising water and unreacted components removed from unreacted component removal apparatus 180 can be introduced into NH₃—CO₂ reactor 160, to increase a conversion to urea by the system 200. Step 40 of method I can thus comprise removing a stream 176 comprising water and unreacted NH₃ and CO₂, and undecomposed ammonium carbamate from the ammonium carbamate decomposition reactor 170 and/or a stream 186 comprising water and unreacted NH₃ and CO₂ from unwanted component removal apparatus 180. Method I can include recycling the stream 176 comprising the water, the unreacted NH₃ and CO₂, and the undecomposed ammonium carbamate and/or the stream 186 comprising water, unreacted NH₃ and unreacted CO₂ to the NH₃—CO₂ reactor 160, to increase a conversion to urea provided by the method.

Method I can further comprise removing additional water 192 from the concentrated aqueous urea 185, or a portion 189 thereof, to provide a semi-solid, molten, and/or solid urea 191. Removing additional water 192 can comprise drying the concentrated aqueous urea 185 or the portion 189 thereof. Accordingly, in embodiments, urea production apparatus 130A of system 200 can further include a drying apparatus 190. Drying apparatus 190 is configured for removing additional water 192 from the concentrated aqueous urea 185 introduced thereto to provide a semi-solid, molten, and/or solid urea 191. The drying apparatus 190 can comprise a vacuum evaporator. Step 40 of method I can thus comprise removing water 192 via drying apparatus 192.

As noted above, method I can further include, at 50, utilizing heat from the collected exhaust gas 115 (e.g., high temperature exhaust gas 115A of FIG. 3) collected at 10 and/or produced during the forming of the urea at 30 in the separating CO₂ from the collected exhaust gas 115 at 20, the forming urea from at least a portion of the separated CO₂ 125 at 30, and/or the separating of one or more components from the urea at 40. For example, system 200 can further include steam production apparatus (e.g., heat exchanger 150 or another heat exchanger) operable to utilize heat produced by the exothermic reaction (Eq. (1)) in the NH₃—CO₂ reactor 160 to produce steam 155. At least a portion of the steam 155A can be utilized as a heat source for decomposing the ammonium carbamate in ammonium carbamate decomposition reactor 170, at least a portion of the steam 155B can be utilized as a heat source for effecting unreacted component removal in unwanted component removal apparatus 180, and/or at least a portion of the steam 155C can be utilized as a heat source in drying apparatus 190.

Accordingly, as depicted in FIG. 3, urea production apparatus 130A of system 200 can further comprise a heat exchanger 150 configured to transfer heat from a high temperature collected exhaust gas 115A (e.g., a collected exhaust gas 115A having a temperature of greater than or equal to about 350, 500, or 700° C.) to produce steam 155 from water 114 introduced into heat exchanger 150 and provide a low temperature exhaust gas 115B (e.g., a collected exhaust gas having a temperature of less than or equal to about 75, 100, or 150° C.) as the collected exhaust gas 115 (FIG. 2). The steam 155 can be utilized in the urea production apparatus 130 (e.g., in ammonium carbamate decomposition reactor 170, in unreacted component removal reactor 180, in drying apparatus 190, or a combination thereof.) In embodiments, heat from the exothermic reaction (e.g., of Eq. (1)) in the urea production apparatus 130 (e.g., produced in NH₃—CO₂ reactor 160) can be utilized in the urea production apparatus 130 (e.g., in ammonium carbamate decomposition reactor 170, in unreacted component removal reactor 180, in drying apparatus 190, or a combination thereof. (For example, the heat exchanger 150 or another heat exchanger) can be utilized to produce steam 155 that can be utilized to heat ammonium carbamate decomposition reactor 170, unreacted component removal reactor 180, and/or drying apparatus 190.)

System 100/200 can thus further include heating apparatus (e.g., heat exchanger 150, etc.) configured for utilizing heat of the collected exhaust gas 115 elsewhere in the system 100/200. For example, system 200 of FIG. 3 can further include heat exchanger 150 configured for utilizing heat of the high temperature collected exhaust gas 115A in the ammonium carbamate decomposition reactor 170, the unreacted component removal apparatus 180, and/or the drying apparatus 190.

In embodiments, method I can further comprise utilizing heat of the collected exhaust gas 115 (e.g., high temperature exhaust gas 115A of FIG. 3) and/or heat of ammonium carbamate solution 165 in the decomposing of the ammonium carbamate, the separating CO₂ from the collected exhaust gas 115, and/or the removing water, unreacted NH₃ and CO₂ stream 186 from the aqueous urea solution 175 to provide the concentrated aqueous urea 185, and/or the drying of all or a portion 189 of the concentrated aqueous urea 185 in drying apparatus 190. Utilizing heat can comprise forming steam 155 and utilizing the steam 155A in the decomposing of the ammonium carbamate, utilizing steam 155 in the separating CO₂ from the collected exhaust gas in separation apparatus 120, utilizing steam 155B in the removing water, unreacted NH₃ and CO₂ stream 186 from the aqueous urea solution 175 to provide the concentrated aqueous urea 185, and/or utilizing steam 155C in the drying in drying apparatus 190.

With reference to FIG. 1, as depicted at step 50, method I can include utilizing heat obtained from the collected exhaust gas 115 obtained at step 10 and/or the forming of the urea at step 30 in the CO₂ separation at step 20, the forming of urea at step 30, and/or the separating of components at step 40. Heat of the collected exhaust gas 115 can be utilized in CO₂ separating step 20, urea forming at step 30 and/or in component removal at step 40 of method I. For example, heat of the collected exhaust gas 115 can be utilized the separating CO₂ 125 from the collected exhaust gas 115 in CO₂ separation apparatus 120 (in CO₂ separating step 20), in the decomposing of the ammonium carbamate in ammonium carbamate decomposition reactor 170 (in urea forming step 30), in component removal in unreacted component removal reactor 180 (in component removal step 40), and/or in drying in drying apparatus 190 (also in component removal step 40). Utilizing heat can comprise forming steam 155 and utilizing the steam 155 for the heating of the various aforementioned processes.

As noted above, method I can further comprise forming diesel exhaust fluid (DEF) at 60. Forming DEF can comprise diluting the urea with water 144 at the wellsite 111 or another wellsite to form diesel exhaust fluid (DEF) 145 for converting toxic nitrogen oxides (NOx) in a diesel combustion exhaust gas of diesel combustion into inert nitrogen gas. The method I can further utilizing the DEF onsite in a diesel engine 113 (e.g., a diesel engine 113 of a diesel truck 114). Forming DEF utilizing at least a portion of the urea produced in urea production apparatus 130 (e.g., urea production apparatus 130A of FIG. 3 and/or urea production apparatus 130B of FIG. 4) at 60 can comprise utilizing the urea product 135 (e.g., aqueous urea solution 175, concentrated aqueous urea 185, or semi-solid, molten, or solid urea 191) as source of urea 136 for preparing DEF 145 to be used in converting toxic nitrogen oxides (NOx) in exhaust gas of diesel combustion into inert N₂ gas.

Accordingly, as depicted in FIG. 2, a system 100 of this disclosure can comprise a DEF production apparatus 140 configured to form the DEF 145 at 60. DEF production apparatus 140 is configured for diluting the urea 165 (e.g., in urea product 135, which can be or comprise aqueous urea solution 175, concentrated aqueous urea 185, and/or semi-solid, molten, or dry urea 191) with water 144 at the wellsite 111 to form diesel exhaust fluid (DEF) 145 for converting toxic nitrogen oxides (NOx) in a diesel combustion exhaust gas of diesel combustion into inert nitrogen gas. The system 100/200 can further comprise one or more onsite diesel engines 113 (e.g., a diesel engine 113 of a diesel truck 114) comprising some of the DEF 145.

Alternatively or additionally, the urea product 135 (e.g., aqueous urea solution 175, concentrated aqueous urea 185, or semi-solid, molten, or solid urea 191) can be utilized as source of liquid or dry fertilizers that can potentially be sold or donated to local farmers.

As depicted in FIG. 2, in embodiments, a system, such as system 100 of FIG. 2 or system 200 of FIG. 3 (or a system 100 or 200 including an electrocatalytic reaction apparatus, such as electrocatalytic reaction apparatus 130B described with regard to FIG. 4 and FIG. 5, as urea production apparatus 130) of this disclosure, or one or more components thereof (e.g., exhaust gas collection apparatus 110, CO₂ separation apparatus 120, urea production apparatus 130, DEF production apparatus 140, or a combination thereof) can be provided on one or more skids 150 (e.g., a trailer skid), whereby at least a portion the separated CO₂ 125 and liquid ammonia 141 can be converted to urea product 135 comprising urea 136 (e.g., aqueous urea solution 175, concentrated aqueous urea 185, semi-solid, molten, or solid urea 191) at the wellsite 111. For example, in embodiments, such as depicted in FIG. 2, urea production apparatus 130, DEF production apparatus 140, or both can be located on one or more skids 150.

Also provided herein is a method comprising: forming urea using, as a reactant, carbon dioxide separated from exhaust gas produced at a wellsite.

The collecting of exhaust gas at 10, the separating of CO₂ at 20, the forming of urea at 30, or a combination thereof can be performed substantially continuously or intermittently.

The system and method of this disclosure can provide for continuous, semi-continuous, or intermittent collecting of exhaust gas 115 from field operating equipment 112 at a wellsite 111 and utilization of the collected exhaust gas 115 to produce urea. The urea can be utilized to benefit at the wellsite 111, for example, as a component of DEF and/or can be sold for profit (e.g., as a fertilizer to farms local or remote from wellsite 111).

Rather than or in addition to injecting downhole separated CO₂ 125 separated from collected exhaust gas 115 for sequestration purposes, separated CO₂ 125 can be converted by the system and disclosure provided herein to convert it, at wellsite 111, to a useful urea product 135 comprising urea 136, such as an aqueous urea solution 175, a concentrated aqueous urea 185, or a semi-solid, molten, or solid urea 191.

Rather than relying on commercial diesel exhaust fluid (DEF) or a commercial urea source for making DEF, an aqueous urea solution can be manufactured at wellsite 111 via the system and method of this disclosure to provide ample supply of DEF for diesel engines of diesel trucks and other apparatus/machinery at the wellsite 111. Aqueous urea solution 175 produced at wellsite 111 can be diluted with dilution water 144 to form DEF 145 for converting toxic nitrogen oxides (NOx) in the exhaust gas of diesel combustion into inert N₂ gas. The DEF can be utilized at wellsite 111 and/or off-site.

In embodiments, the system of this disclosure can be provided on a skid (e.g., a trailer skid), whereby at least a portion the separated CO₂ 125 and liquid ammonia 141 can be converted to urea product 135 comprising urea 136 (e.g., aqueous urea solution 175, concentrated aqueous urea 185, semi-solid, molten, or solid urea 191) at the wellsite 111.

In embodiments, the heat of collected exhaust gas 115 can be utilized in providing a heat source utilized during the separating of one or more components from the urea at step 40 of method I.

In embodiments, at least a portion of the system 100 (e.g., urea production apparatus 130, DEF production apparatus 140) is provided as a small-scale urea plant (e.g., on one or more skids 150) at wellsite 111, whereby urea product 135 (e.g., aqueous urea solution 175, concentrated aqueous urea 185, or dry urea 191) can be produced on location, DEF can be produced on location (e.g., and utilized in diesel trucks or other diesel engines), and/or a source of liquid urea fertilizer (or dry fertilizer) can be provided and sold or donated to farmers (e.g., local farmers).

### Additional Disclosure

The following are non-limiting, specific embodiments in accordance with the present disclosure:

In a first embodiment, a method comprises collecting exhaust gas comprising carbon dioxide (CO₂) at a wellsite to provide a collected exhaust gas; separating CO₂ from the collected exhaust gas to provide a separated CO₂; and forming urea utilizing at least a portion of the separated CO₂.

A second embodiment can include the method of the first embodiment, wherein forming urea comprises reacting the separated CO₂ with ammonia (NH₃) to form ammonium carbamate and decomposing the ammonium carbamate to form the urea; or wherein forming urea comprises reacting the separated CO₂ directly with nitrogen (N₂) gas to form the urea.

A third embodiment can include the method of the first or the second embodiment, wherein forming urea comprises reacting the at least the portion of the separated CO₂ with ammonia to form ammonium carbamate and decomposing the ammonium carbamate to form the urea.

A fourth embodiment can include the method of the third embodiment, wherein forming urea further comprises: compressing the at least a portion of the separated CO₂ to provide a compressed CO₂; reacting the compressed CO₂ with the ammonia to form an ammonium carbamate solution comprising the ammonium carbamate; and decomposing the ammonium carbamate to provide an aqueous urea solution comprising the urea.

A fifth embodiment can include the method of the fourth embodiment further comprising removing water, and unreacted NH₃ and CO₂ from the aqueous urea solution to provide a concentrated aqueous urea.

A sixth embodiment can include the method of the fifth embodiment, further comprising utilizing heat of the collected exhaust gas in the decomposing of the ammonium carbamate, the separating CO₂ from the collected exhaust gas, and/or the removing water, unreacted NH₃ and CO₂ from the aqueous urea solution to provide the concentrated aqueous urea.

A seventh embodiment can include the method of the sixth embodiment, wherein utilizing heat further comprises forming steam and utilizing the steam in the decomposing of the ammonium carbamate, the separating CO₂ from the collected exhaust gas, and/or the removing water, unreacted NH₃ and CO₂ from the aqueous urea solution to provide the concentrated aqueous urea.

An eighth embodiment can include the method of any one of the fifth to seventh embodiments, wherein removing the water and unreacted NH₃ and CO₂ is effected via a flash chamber.

A ninth embodiment can include the method of any one of the fifth to eighth embodiments further comprising removing additional water from the concentrated aqueous urea to provide a semi-solid, molten, and/or solid urea.

A tenth embodiment can include the method of the ninth embodiment, wherein removing additional water comprises drying the concentrated aqueous urea in a vacuum evaporator.

An eleventh embodiment can include the method of any one of the fourth to tenth embodiments, wherein compressing comprises compressing to a pressure of greater than or equal to about 130, 140, or 150 atmospheres (13.2, 14.2, or 15.2 MPa), and/or in a range of from about 130 to about 150 atm (from about 13.2 to about 15.2 MPa).

A twelfth embodiment can include the method of any one of the fourth to eleventh embodiments, wherein reacting the compressed CO₂ and ammonia to form the ammonium carbamate solution comprises introducing the compressed CO₂ and liquid ammonia into an NH₃—CO₂ reactor, whereby the compressed CO₂ and the liquid NH₃ react via an exothermic reaction to produce the ammonium carbamate.

A thirteenth embodiment can include the method of the twelfth embodiment, wherein the NH₃—CO₂ reactor has an operating temperature in a range of from about 350° F. to about 390° F. (from about 176.7° C. to about 198.9° C.), from about 360° F. to about 390° F. (from about 182.2° C. to about 198.9° C.), from about 370° F. to about 390° F. (from about 176.7° C. to about 198.9° C.), or greater than or equal to about 350° F., 360° F., 370° F., 380° F., or 390° F. (greater than or equal to about 176.7° C., 182.2° C., 187.8° C., 193.3° C., or 198.8° C.), and/or a pressure in a range of from about 130 atmospheres (atm) to about 160 atm (from about 13.2 MPa to about 16.2MPa), from about 140 atm to about 160 atm (from about 14.2 MPa to about 16.2MPa), from about 150 atm to about 160 atm (from about 15.2 MPa to about 16.2MPa), or greater than or equal to about 130, 140, 150, 160, or 170 atm (greater than or equal to about 13.2, 14.2, 15.2, 16.2, or 17.2 MPa).

A fourteenth embodiment can include the method of the thirteenth embodiment further comprising using heat produced by the exothermic reaction in the NH₃—CO₂ reactor to produce steam.

A fifteenth embodiment can include the method of the fourteenth embodiment further comprising using the steam as a heat source for decomposing the ammonium carbamate.

A sixteenth embodiment can include the method of any one of the fourth to fifteenth embodiments, wherein decomposing the ammonium carbamate further comprises introducing the ammonium carbamate into an ammonium carbamate decomposition reactor, whereby the ammonium carbamate decomposes to provide the urea of the aqueous urea solution via a reduction in the temperature and the pressure of the ammonium carbamate in the ammonium carbamate decomposition reactor.

A seventeenth embodiment can include the method of the sixteenth embodiment, wherein the ammonium carbamate decomposition reactor is and/or is operated as a distillation column.

An eighteenth embodiment can include the method of any one of the sixteenth to seventeenth embodiments, wherein decomposing further comprises reducing the temperature and pressure of the ammonium carbamate in the ammonium carbonate decomposition reactor to a temperature in a range of from about 250° F. to about 280° F. (from about 121.1° C. to about 137.8° C.), from about 260° F. to about 280° F. (from about 126.7° C. to about 137.8° C.), from about 270° F. to about 280° F. (from about 132.2° C. to about 137.8° C.), or less than or equal to about 250° F., 260° F., 270° F., 280° F., or 290° F. (less than or equal to about 121.1° C., 126.7° C., 132.2° C., 137.8° C., or 143.3° C.), and/or a pressure in a range of from about 20 atmospheres (atm) to about 50 atm (from about 2.1 MPa to about 5.1 MPa), from about 25 atm to about 45 atm (from about 2.5 MPa to about 4.6 MPa), from about 30 atm to about 50 atm (from about 3.0 MPa to about 5.1 MPa), or less than or equal to about 50, 45, 40, 35, or 30 atm (less than or equal to about 5.1, 4.6, 4.1, 3.5, or 3.0 MPa).

A nineteenth embodiment can include the method of any one of the sixteenth to eighteenth embodiments further comprising removing a stream comprising water and unreacted NH₃ and CO₂, and undecomposed ammonium carbamate from the ammonium carbamate decomposition reactor.

A twentieth embodiment can include the method of the nineteenth embodiment, further comprising recycling the stream comprising the water, the unreacted NH₃ and CO₂, and the undecomposed ammonium carbamate to the NH₃—CO₂ reactor, to increase a conversion to urea.

A twenty first embodiment can include the method of any one of the fourth to twentieth embodiments further comprising utilizing heat of the collected exhaust gas in the decomposing of the ammonium carbamate and/or the separating CO₂ from the collected exhaust gas.

A twenty second embodiment can include the method of the twenty first embodiment, wherein utilizing heat further comprises forming steam and utilizing the steam in the decomposing of the ammonium carbamate and/or the separating CO₂ from the collected exhaust gas.

A twenty third embodiment can include the method of any one of the second to twenty second embodiments, comprising forming urea by reacting the separated CO₂ directly with nitrogen gas to form the urea.

A twenty fourth embodiment can include the method of the twenty third embodiment further comprising forming the urea by converting nitrogen gas and the at least the portion of the separated CO₂ directly into the urea in aqueous solution via an electrocatalytic reaction.

A twenty fifth embodiment can include the method of the twenty fourth embodiment, wherein the electrocatalytic reaction occurs at ambient temperature and pressure (e.g., a temperature of about 77° F. (21° C.) and a pressure of about atmospheric pressure (101 kPa).

A twenty sixth embodiment can include the method of any one of the twenty fourth to twenty fifth embodiments, wherein the electrocatalytic reaction is effected in the presence of a catalyst comprising palladium-copper (Pd—Cu) nanoparticles on titanium dioxide (TiO₂) nanosheets.

A twenty seventh embodiment can include the method of the twenty sixth embodiment, wherein the electrocatalytic reaction is carried out in a flow reactor cell comprising a cathode of carbon paper loaded with the catalyst and a nickel based anode, separated by a membrane in a chamber filled with an aqueous potassium bicarbonate electrolyte.

A twenty eighth embodiment can include the method of the twenty seventh embodiment further comprising pumping the N₂ gas and the at least the portion of the separated CO₂ gas through the flow reactor cell so both the nitrogen gas and the CO₂ gas are adsorbed on the catalyst and react to produce the urea.

A twenty ninth embodiment can include the method of any one of the first to twenty eighth embodiments further comprising diluting the urea with water at the wellsite to form diesel exhaust fluid (DEF) for converting toxic nitrogen oxides (NOx) in a diesel combustion exhaust gas of diesel combustion into inert nitrogen gas.

A thirtieth embodiment can include the method of the twenty ninth embodiment further comprising utilizing the DEF onsite in a diesel engine (e.g., a diesel engine of a diesel truck).

In a thirty first embodiment, a system comprises: an exhaust gas collection system configured for collecting exhaust gas comprising carbon dioxide (CO₂) at a wellsite to provide a collected exhaust gas; a CO₂ separation apparatus configured for separating CO₂ from the collected exhaust gas to provide a separated CO₂; and a urea production apparatus configured for forming urea utilizing at least a portion of the separated CO₂.

A thirty second embodiment can include the system of the thirty first embodiment, wherein the urea production apparatus comprises a NH₃—CO₂ reactor configured for reacting the separated CO₂ with ammonia (NH₃) to form ammonium carbamate and an ammonium carbamate decomposition reactor configured for decomposing the ammonium carbamate to form the urea, or wherein the urea production apparatus comprises an electrocatalytic reactor configured for forming the urea by reacting the separated CO₂ directly with nitrogen gas.

A thirty third embodiment can include the system of the thirty second embodiment, wherein the urea production apparatus comprises the NH₃—CO₂ reactor and the ammonium carbamate decomposition reactor configured for decomposing the ammonium carbamate, wherein the NH₃—CO₂ reactor is configured for reacting the separated CO₂ with liquid ammonia comprising the NH₃ to form the ammonium carbamate and wherein the ammonium carbamate decomposition reactor is configured for decomposing the ammonium carbamate to form an aqueous urea solution comprising the urea.

A thirty fourth embodiment can include the system of the thirty third embodiment further comprising: a compressor configured for compressing the at least a portion of the separated CO₂ to provide a compressed CO₂, wherein the NH₃—CO₂ reactor is configured for reacting the compressed CO₂ with the liquid ammonia via an exothermic reaction to form the ammonium carbamate; and an unreacted component removal apparatus (e.g., a flash chamber) configured for separating water and unreacted CO₂ and NH₃ from the aqueous urea solution provided in the ammonium carbamate reactor to provide a concentrated aqueous urea having a higher concentration of urea than the aqueous urea solution.

A thirty fifth embodiment can include the system of the thirty fourth embodiment further comprising a drying apparatus configured for removing additional water from the concentrated aqueous urea to provide a semi-solid, molten, and/or solid urea.

A thirty sixth embodiment can include the system of the thirty fifth embodiment, wherein the drying apparatus comprises a vacuum evaporator.

A thirty seventh embodiment can include the method of any one of the thirty fourth to thirty sixth embodiments, wherein the compressor is operable to compress the at least the portion of the separated CO₂ to a pressure of greater than or equal to about 130, 140, or 150 atmospheres (13.2, 14.2, or 15.2 MPa), and/or in a range of from about 130 to about 150 atm (from about 13.2 to about 15.2 MPa).

A thirty eighth embodiment can include the system of any one of the thirty fourth to thirty seventh embodiments, wherein reacting the compressed CO₂ and the liquid ammonia to form the ammonium carbamate comprises introducing the compressed CO₂ and liquid ammonia into the NH₃—CO₂ reactor, whereby the compressed CO₂ and the liquid NH₃ react via an exothermic reaction to form the ammonium carbamate.

A thirty ninth embodiment can include the system of the thirty eighth embodiment, wherein the NH₃—CO₂ reactor has an operating temperature in a range of from about 350° F. to about 390° F. (from about 176.7° C. to about 198.9° C.), from about 360° F. to about 390° F. (from about 182.2° C. to about 198.9° C.), from about 370° F. to about 390° F. (from about 176.7° C. to about 198.9° C.), or greater than or equal to about 350° F., 360° F., 370° F., 380° F., or 390° F. (greater than or equal to about 176.7° C., 182.2° C., 187.8° C., 193.3° C., or 198.8° C.), and/or a pressure in a range of from about 130 atmospheres (atm) to about 160 atm (from about 13.2 MPa to about 16.2MPa), from about 140 atm to about 160 atm (from about 14.2 MPa to about 16.2MPa), from about 150 atm to about 160 atm (from about 15.2 MPa to about 16.2MPa), or greater than or equal to about 130, 140, 150, 160, or 170 atm (greater than or equal to about 13.2, 14.2, 15.2, 16.2, or 17.2 MPa).

A fortieth embodiment can include the system of any one of the thirty eighth to thirty ninth embodiments further comprising steam production apparatus operable to utilize heat produced by the exothermic reaction in the NH₃—CO₂ reactor to produce steam.

A forty first embodiment can include the system of the fortieth embodiment further comprising a steam line from the NH₃—CO₂ reactor to the ammonium carbamate decomposition reactor, whereby the at least a portion of the steam is utilized as a heat source for decomposing the ammonium carbamate.

A forty second embodiment can include the system of any one of the thirty second to forty first embodiments, wherein the ammonium carbamate decomposition reactor is operable to decompose the ammonium carbamate to provide the aqueous urea solution via a reduction in the temperature and pressure of the ammonium carbamate in the ammonium carbamate decomposition reactor.

A forty third embodiment can include the system of the forty second embodiment, wherein the ammonium carbamate decomposition reactor comprises a distillation column.

A forty fourth embodiment can include the system of any one of the forty second to forty third embodiments, wherein the ammonium carbamate decomposition reactor effects the decomposing via a reduction of the temperature and pressure of the ammonium carbamate therein to a temperature in a range of from about 250° F. to about 280° F. (from about 121.1° C. to about 137.8° C.), from about 260° F. to about 280° F. (from about 126.7° C. to about 137.8° C.), from about 270° F. to about 280° F. (from about 132.2° C. to about 137.8° C.), or less than or equal to about 250° F., 260° F., 270° F., 280° F., or 290° F. (less than or equal to about 121.1° C., 126.7° C., 132.2° C., 137.8° C., or 143.3° C.), and/or a pressure in a range of from about 20 atmospheres (atm) to about 50 atm (from about 2.1 MPa to about 5.1 MPa), from about 25 atm to about 45 atm (from about 2.5 MPa to about 4.6 MPa), from about 30 atm to about 50 atm (from about 3.0 MPa to about 5.1 MPa), or less than or equal to about 50, 45, 40, 35, or 30 atm (less than or equal to about 5.1, 4.6, 4.1, 3.5, or 3.0 MPa).

A forty fifth embodiment can include the system of any one of the thirty second to forty fourth embodiments, wherein the ammonium carbamate decomposition reactor provides a stream comprising water, unreacted NH₃ and CO₂, and undecomposed ammonium carbamate.

A forty sixth embodiment can include the system of the forty fifth embodiment, wherein the ammonium carbamate decomposition reactor is fluidly connected with the NH₃—CO₂ reactor, such that the stream comprising unreacted NH₃ and CO₂, and undecomposed ammonium carbamate is recycled to the NH₃—CO₂ reactor, to increase a conversion to urea by the system.

A forty seventh embodiment can include the system of any one of the thirty third to forty sixth embodiments further comprising an unreacted component removal apparatus configured for removing water and unreacted NH₃ and CO₂ from the aqueous urea solution to provide a concentrated aqueous urea.

A forty eighth embodiment can include the system of the forty seventh embodiment, wherein the unreacted component removal apparatus comprises a flash chamber.

A forty ninth embodiment can include the system of any one of the thirty third to forty eighth embodiments further comprising heating apparatus configured for utilizing heat of the collected exhaust gas in the ammonium carbamate decomposition reactor and/or the unreacted component removal apparatus.

A fiftieth embodiment can include the system of the forty ninth embodiment, wherein the heating apparatus comprises steam generating apparatus, and wherein the steam is utilized for heating in the ammonium carbamate decomposition reactor and/or the unreacted component removal apparatus.

A fifty first embodiment can include the system of any one of the forty ninth or fiftieth embodiments further comprising a drying apparatus configured for removing additional water from the concentrated aqueous urea to provide a semi-solid, molten, and/or solid urea, and wherein the heating apparatus is configured for utilizing the heat of the collected exhaust gas in the ammonium carbamate decomposition reactor, the unreacted component removal apparatus, the drying apparatus, or a combination thereof.

A fifty second embodiment can include the system of any one of the thirty second to fifty first embodiments, wherein the urea production apparatus comprises the electrocatalytic apparatus configured for forming the urea by reacting the separated CO₂ directly with nitrogen gas to form the urea.

A fifty third embodiment can include the system of the fifty second embodiment, wherein the electrocatalytic apparatus is configured for forming the urea by converting nitrogen gas and the at least the portion of the separated CO₂ directly into urea in water via an electrocatalytic reaction.

A fifty fourth embodiment can include the system of the fifty third embodiment, wherein the electrocatalytic apparatus is configured to effect the electrocatalytic reaction at about ambient temperature and pressure (e.g., a temperature of about 77° F. (21° C.) and atmospheric pressure (101 kPa).

A fifty fifth embodiment can include the system of the fifty fourth embodiment, wherein the electrocatalytic reactor comprises a catalyst comprising palladium-copper (Pd—Cu) nanoparticles on titanium dioxide (TiO₂) nanosheets.

A fifty sixth embodiment can include the system of the fifty fifth embodiment, wherein the electrocatalytic reactor comprises a flow reactor cell comprising a cathode of carbon paper loaded with the catalyst and a nickel based anode, separated by a membrane in a chamber filled with an aqueous potassium bicarbonate electrolyte, wherein the urea is formed by pumping the N₂ gas and the at least the portion of the separated CO₂ gas through the flow reactor cell so both the nitrogen gas and the CO₂ gas are adsorbed on the catalyst and react to produce the urea.

A fifty seventh embodiment can include the system of any one of the thirty first to fifty sixth embodiments further comprising DEF production apparatus configured for diluting the urea with water at the wellsite to form diesel exhaust fluid (DEF) for converting toxic nitrogen oxides (NOx) in a diesel combustion exhaust gas of diesel combustion into inert nitrogen gas.

A fifty eighth embodiment can include the system of the fifty seventh embodiment further comprising an onsite diesel engine (e.g., a diesel engine of a diesel truck) comprising some of the DEF.

In a fifty ninth embodiment, a method comprises: producing urea a detailed herein using, as a reactant, carbon dioxide separated from exhaust gas produced at a wellsite.

A sixtieth embodiment can include the method of the fifty ninth embodiment, wherein the producing is performed substantially continuously or intermittently.

While embodiments have been shown and described, modifications thereof can be made by one skilled in the art without departing from the spirit and teachings of this disclosure. The embodiments described herein are exemplary only, and are not intended to be limiting. Many variations and modifications of the embodiments disclosed herein are possible and are within the scope of this disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, RI, and an upper limit, Ru, is disclosed, any number falling within the range is specifically disclosed. In particular, the following numbers within the range are specifically disclosed: R=R1+k* (Ru−R1), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of broader terms such as comprises, includes, having, etc. should be understood to provide support for narrower terms such as consisting of, consisting essentially of, comprised substantially of, etc. When a feature is described as “optional,” both embodiments with this feature and embodiments without this feature are disclosed. Similarly, the present disclosure contemplates embodiments where this “optional” feature is required and embodiments where this feature is specifically excluded.

Accordingly, the scope of protection is not limited by the description set out above but is only limited by the claims which follow, that scope including all equivalents of the subject matter of the claims. Each and every claim is incorporated into the specification as embodiments of the present disclosure. Thus, the claims are a further description and are an addition to the embodiments of the present disclosure. The discussion of a reference herein is not an admission that it is prior art, especially any reference that can have a publication date after the priority date of this application. The disclosures of all patents, patent applications, and publications cited herein are hereby incorporated by reference, to the extent that they provide exemplary, procedural, or other details supplementary to those set forth herein.

What is claimed is:

1. A method comprising:
collecting exhaust gas comprising carbon dioxide (CO₂) at a wellsite to provide a collected exhaust gas;
separating CO₂ from the collected exhaust gas to provide a separated CO₂; and
forming urea utilizing at least a portion of the separated CO₂.

2. The method of claim 1, wherein forming urea comprises reacting the separated CO₂ with ammonia (NH₃) to form ammonium carbamate and decomposing the ammonium carbamate to form the urea; or wherein forming urea comprises reacting the separated CO₂ directly with nitrogen (N₂) gas to form the urea.

3. The method of claim 2, wherein forming urea comprises reacting the at least the portion of the separated CO₂ with ammonia to form ammonium carbamate and decomposing the ammonium carbamate to form the urea.

4. The method of claim 3, wherein forming urea further comprises:
compressing the at least a portion of the separated CO₂ to provide a compressed CO₂;
reacting the compressed CO₂ with the ammonia to form an ammonium carbamate solution comprising the ammonium carbamate; and
decomposing the ammonium carbamate to provide an aqueous urea solution comprising the urea.

5. The method of claim 4 further comprising removing water, and unreacted NH₃ and CO₂ from the aqueous urea solution to provide a concentrated aqueous urea.

6. The method of claim 5 further comprising utilizing heat of the collected exhaust gas in the decomposing of the ammonium carbamate, the separating CO₂ from the collected exhaust gas, and/or the removing water, unreacted NH₃ and CO₂ from the aqueous urea solution to provide the concentrated aqueous urea.

7. The method of claim 6, wherein utilizing heat further comprises forming steam and utilizing the steam in the decomposing of the ammonium carbamate, the separating CO₂ from the collected exhaust gas, and/or the removing water, unreacted NH₃ and CO₂ from the aqueous urea solution to provide the concentrated aqueous urea.

8. The method of claim 4, wherein decomposing the ammonium carbamate further comprises introducing the ammonium carbamate into an ammonium carbamate decomposition reactor, whereby the ammonium carbamate decomposes to provide the urea of the aqueous urea solution via a reduction in the temperature and the pressure of the ammonium carbamate in the ammonium carbamate decomposition reactor.

9. The method of claim 2, comprising forming urea by reacting the separated CO₂ directly with nitrogen gas to form the urea.

10. The method of claim 9 further comprising forming the urea by converting nitrogen gas and the at least the portion of the separated CO₂ directly into the urea in aqueous solution via an electrocatalytic reaction.

11. The method of claim 1 further comprising diluting the urea with water at the wellsite to form diesel exhaust fluid (DEF) for converting toxic nitrogen oxides (NOx) in a diesel combustion exhaust gas of diesel combustion into inert nitrogen gas.

12. A system comprising:
an exhaust gas collection system configured for collecting exhaust gas comprising carbon dioxide (CO₂) at a wellsite to provide a collected exhaust gas;
a CO₂ separation apparatus configured for separating CO₂ from the collected exhaust gas to provide a separated CO₂; and
a urea production apparatus configured for forming urea utilizing at least a portion of the separated CO₂.

13. The system of claim 12, wherein the urea production apparatus comprises a NH₃—CO₂ reactor configured for reacting the separated CO₂ with ammonia (NH₃) to form ammonium carbamate and an ammonium carbamate decomposition reactor configured for decomposing the ammonium carbamate to form the urea, or wherein the urea production apparatus comprises an electrocatalytic reactor configured for forming the urea by reacting the separated CO₂ directly with nitrogen gas.

14. The system of claim 13, wherein the urea production apparatus comprises the NH₃—CO₂ reactor and the ammonium carbamate decomposition reactor configured for decomposing the ammonium carbamate, wherein the NH₃—CO₂ reactor is configured for reacting the separated CO₂ with liquid ammonia comprising the NH₃ to form the ammonium carbamate and wherein the ammonium carbamate decomposition reactor is configured for decomposing the ammonium carbamate to form an aqueous urea solution comprising the urea.

15. The system of claim 14 further comprising:
a compressor configured for compressing the at least a portion of the separated CO₂ to provide a compressed CO₂, wherein the NH₃—CO₂ reactor is configured for reacting the compressed CO₂ with the liquid ammonia via an exothermic reaction to form the ammonium carbamate; and
an unreacted component removal apparatus configured for separating water and unreacted CO₂ and NH₃ from the aqueous urea solution provided in the ammonium carbamate reactor to provide a concentrated aqueous urea having a higher concentration of urea than the aqueous urea solution.

16. The system of claim 13, wherein the urea production apparatus comprises the electrocatalytic apparatus configured for forming the urea by reacting the separated CO₂ directly with nitrogen gas to form the urea.

17. The system of claim 16, wherein the electrocatalytic apparatus is configured for forming the urea by converting nitrogen gas and the at least the portion of the separated CO₂ directly into urea in water via an electrocatalytic reaction.

18. The system of claim 12 further comprising DEF production apparatus configured for diluting the urea with water at the wellsite to form diesel exhaust fluid (DEF) for converting toxic nitrogen oxides (NOx) in a diesel combustion exhaust gas of diesel combustion into inert nitrogen gas.

19. A method comprising:
producing urea using, as a reactant, carbon dioxide separated from exhaust gas produced at a wellsite.

20. The method of claim 19, wherein the producing is performed substantially continuously or intermittently.
